(19) United States
(12) Patent Application Publication
(10) Pub. No.: US 2026/0260700 A1
(43) Pub. Date: Sep. 3, 2026

(54) METHODS FOR DETERMINING AN ARM ANEUPLOIDY SCORE

(71) Applicant: Life Technologies Corporation, Carlsbad, CA (US)

(72) Inventors: Ying Jin, Los Gatos, CA (US); Mohit Gupta, Dublin, CA (US); Seth Sadis, Ann Arbor, MI (US)

(21) Appl. No.: 19/652,196

(22) Filed: Apr. 20, 2026

Related U.S. Application Data
(63) Continuation of application No. PCT/US2024/052564, filed on Oct. 23, 2024.
(60) Provisional application No. 63/592,732, filed on Oct. 24, 2023.

(51) Int. Cl. G16B 20/20; C12Q 1/6886; G16B 20/10

(52) U.S. Cl. CPC G16B 20/20; C12Q 1/6886; G16B 20/10; C12Q 2600/156

(57) ABSTRACT

Methods for determining an arm aneuploidy score in a tumor sample genome include selectively amplifying nucleic acid sequences at specific locations in the tumor genome using a targeted panel to generate sequence reads. Next, divide the genome locations into segments with homogeneous copy numbers based on log odds of heterozygous SNPs and CNV log ratios of the sequence reads. Identify gain and loss segments relative to a reference copy number and intersecting respective chromosome arms. Compare the cellularities of these segments to a minimum threshold. Retain the longest segment for the arm that meets the minimum cellularity and sum its total bases. Divide this total by the number of bases in the arm to yield a fraction. If this fraction meets a minimum threshold, filter the segment based on fold changes and determine gains or losses. Count the arms with called gains or losses to generate the arm aneuploidy score.

### CROSS-REFERENCE TO RELATED APPLICATIONS

This application is a continuation application under 35 U.S.C. 119 (a)-(d) of pending International Application no. PCT/US 2024/052564, filed Oct. 23, 2024, which claims the benefit under 35 U.S.C. § 119 (e) of U.S. Provisional Application no. U.S. 63/592,732, filed Oct. 24, 2023. The entire contents of the aforementioned applications are incorporated by reference herein.

### FIELD

The present disclosure relates to methods, systems, and computer-readable media for determining an arm aneuploidy score, and, more specifically, to methods, systems, and computer-readable media for determining an arm aneuploidy score in a tumor sample genome using nucleic acid sequencing data from targeted sequencing panels and next-generation sequencing (NGS) technology.

### BRIEF DESCRIPTION OF THE DRAWINGS

FIGS. 1A and 1B are block diagrams of an example process for analyzing a sample genome to determine an arm aneuploidy score.

FIG. 2 is a schematic diagram of an exemplary system for reconstructing a nucleic acid sequence, in accordance with various embodiments.

FIG. 3 is an example of a block diagram of an analysis pipeline for signal data obtained from a nucleic acid sequencing instrument.

### DETAILED DESCRIPTION

In accordance with the teachings and principles embodied in this application, new methods, systems and non-transitory machine-readable storage medium are provided to determine an arm aneuploidy score by analysis of nucleic acid sequence reads from a tumor sample genome.

In various embodiments, DNA (deoxyribonucleic acid) may be referred to as a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. In various embodiments, “nucleic acid sequencing data,” “nucleic acid sequencing information,” “nucleic acid sequence,” “genomic sequence,” “genetic sequence,” or “fragment sequence,” “nucleic acid sequence read” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, for example 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” or NGS refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization.

The phrase “genomic variants” or “genome variants” denote a single or a grouping of sequences (in DNA or RNA) that have undergone changes as referenced against a particular species or sub-populations within a particular species due to mutations, recombination/crossover or genetic drift. Examples of types of genomic variants include, but are not limited to: single nucleotide polymorphisms (SNPs), copy number variations (CNVs), insertions/deletions (Indels), inversions, etc.

In various embodiments, genomic variants can be detected using a nucleic acid sequencing system and/or analysis of sequencing data. The sequencing workflow can begin with the test sample being sheared or digested into hundreds, thousands or millions of smaller fragments which are sequenced on a nucleic acid sequencer to provide hundreds, thousands or millions of sequence reads, such as nucleic acid sequence reads. Each read can then be mapped to a reference or target genome, and in the case of mate-pair fragments, the reads can be paired thereby allowing interrogation of repetitive regions of the genome. The results of mapping and pairing can be used as input for various standalone or integrated genome variant (for example, SNP, CNV, Indel, inversion, etc.) analysis tools.

The phrase “sample genome” can denote a whole or partial genome of an organism.

The term “allele” as used herein refers to a genetic variation associated with a gene or a segment of DNA, i.e., one of two or more alternate forms of a DNA sequence occupying the same locus.

The term “locus” as used herein refers to a specific position on a chromosome or a nucleic acid molecule. Alleles of a locus are located at identical sites on homologous chromosomes.

As used herein, a “targeted panel” refers to a set of target-specific primers that are designed for selective amplification of target gene sequences in a sample. In some embodiments, following selective amplification of at least one target sequence, the workflow further includes nucleic acid sequencing of the amplified target sequence.

As used herein, “target sequence” or “target gene sequence” and its derivatives, refer to any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

As used herein, “target-specific primer” and its derivatives, refers to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, a forward target-specific primer and a reverse target-specific primer define a target-specific primer pair that can be used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence.

A targeted panel with low sample input requirements may be used to determine an arm aneuploidy score for a tumor sample. A targeted panel may provide a viable alternative to whole genome sequencing that may have higher input sample requirements. In some embodiments, the targeted panel may comprise the Oncomine™ Comprehensive Assay Plus, or OCA Plus panel, (Thermo Fisher Scientific). The OCA Plus panel interrogates 502 cancer-related genes. The OCA Plus panel has approximately 13,473 amplicons, including 1889 amplicons designed specifically to include heterozygous SNPs that have high minor allele frequencies and are spread evenly across the genome, including chromosome arms. In addition, the heterozygous SNPs present in the targeted medical content of the panel are also used. The panel heterozygous SNPs allows comprehensive profiling of the tumor sample for structural alterations and copy number (CN) changes. A total of 19 “p” arms and 23 “q” arms are covered by the OCA Plus panel. The chromosome arms covered include: 1p, 1q, 2p, 2q, 3p, 3q, 4p, 4q, 5p, 5q, 6p, 6q, 7p, 7q, 8p, 8q, 9p, 9q, 10p, 10q, 11p, 11q, 12p, 12q, 13q, 14q, 15q, 16p, 16q, 17p, 17q, 18p, 18q, 19p, 19q, 20p, 20q, 21p, 21q, 22q, Xp, Xq. The OCA Plus panel may use a recommended amount of 20 ng, and as little as 10 ng, of nucleic acid isolated from formaldehyde fixed paraffin embedded (FFPE) tumor samples including fine needle biopsies. In some embodiments, the panel may comprise a custom targeted panel or other targeted panel that include coverage of the “p” arms and “q” arms.

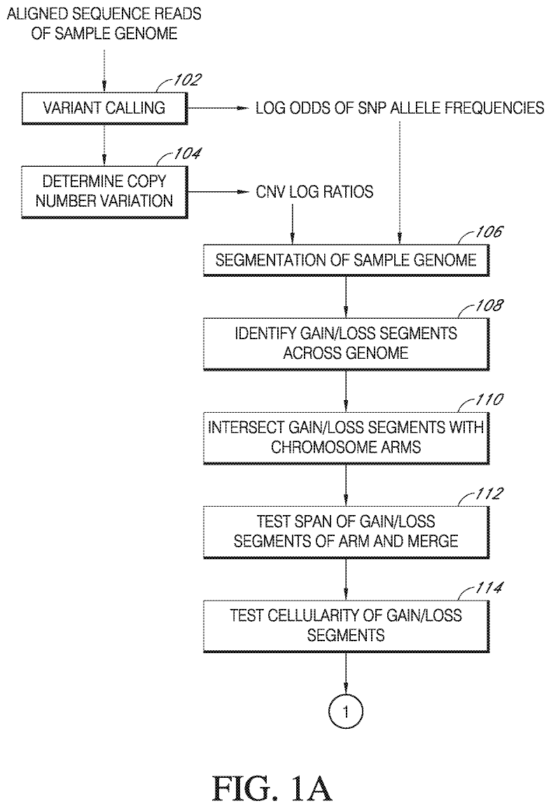

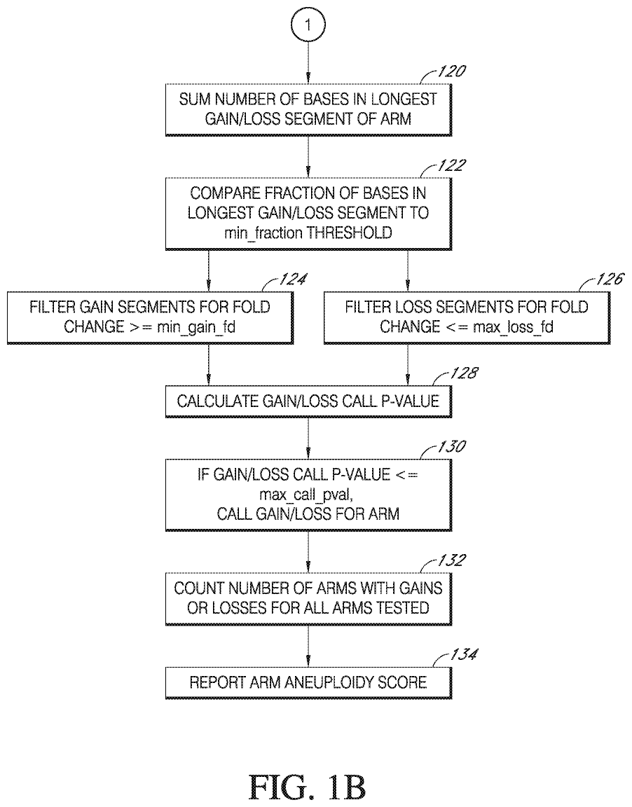

FIGS. 1A and 1B are block diagrams of an example process for analyzing a sample genome to determine an arm aneuploidy score. Selectively amplifying nucleic acid sequences at targeted locations in the tumor sample genome by a targeted panel with a low sample input from the tumor sample and sequencing by a nucleic acid sequencing instrument generates a plurality of nucleic acid sequence reads. The sequence reads are mapped to a reference genome to produce the aligned sequence reads. In the variant calling step 102, a processor receives aligned sequence reads resulting from targeted sequencing of the tumor sample. The aligned sequence reads can be retrieved from a file using a BAM file format, for example. The aligned sequence reads may correspond to a plurality of targeted locations in the tumor sample genome. The variant calling step 102 may be configured by one or more variant caller parameters. The variant calling step 102 may provide an observed population of variants, such as SNPs (single nucleotide polymorphism), detected in the aligned sequence reads. In addition, the variant calling step 102 may determine the log odds for variant allele frequency of observed population of SNPs. The log odds is calculated as the natural logarithm of the ratio of the number of sequence reads with the variant allele to the number of sequence reads with the reference allele. In some embodiments, the variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, each of which incorporated by reference herein in its entirety. Other variant detection methods may be used. In various embodiments, a variant caller can be configured to communicate variants called for a sample genome as a *vcf, *gff, or *hdf data file. The called variant information can be communicated using any file format as long as the called variant information can be parsed and/or extracted for analysis. The copy number variation (CNV) step 104 may provide copy-number estimates and CNV log ratios of the aligned sequence reads. The CNV log ratios are calculated as the log 2 ratios of the copy-number estimates relative to the baseline copy number for each amplicon in the assay. In some embodiments, the CNV detection methods for use with the present teachings may include one or more features described in U.S. Pat. Appl. Publ. No. 2018/0268103, published Sep. 20, 2018, U.S. Pat. Appl. Publ. No. 2014/0256571, published Sep. 11, 2014, and U.S. Pat. Appl. Publ. No. 2016/0103957, published Apr. 14, 2016, each of which incorporated by reference herein in its entirety. Other CNV detection methods may be used. The segmentation step 106 uses the log odds and the CNV log ratios to divide the genome sequence into segments having homogeneous copy numbers. For example, the OCA Plus panel provides 1889 amplicons with heterozygous SNPs designed specifically to cover the genome and segment it for CN changes using joint segmentation of CNV log ratios and allelic log odds. The segmentation algorithm is circular binary segmentation that aims for joint segmentation of log 2 ratios and log odds to detect change points using Hotelling T2 statistic. (See e.g., R. Shen et al., FACETS: allele-specific copy number and clonal heterogeneity analysis tool for high-throughput DNA sequencing, Nucleic Acids Research, 2016, Vol. 44, No. 16 e131 doi: 10.1093/nar/gkw520). Optionally, the segmentation step 106, may exclude segments that have fewer than a minimum number of heterozygous SNPs. For example, the minimum number of SNPs in the segment may be set to a number in the range of 5 to 15 SNPs.

In step 108, the segments across the genome with CN changes of gain or loss (referred to as gain/loss segment herein), with respect to a reference copy number (ref_cn) adjusted by an informatically derived baseline for the sample are identified. Methods for deriving a baseline for CNV detection for use with the present teachings may include one or more features described in U.S. Pat. Appl. Publ. No. 2014/0256571, published Sep. 11, 2014. In step 110, the gain/loss segments having locations that intersect chromosome arms are identified. In step 112, the span of gain/loss segments for each arm are tested and possibly merged. Segments may be merged in the situation where two segments with identical copy numbers are separated by a segment with a different copy number, as follows:
a) Locate two segments within the arm that have the same copy number, where the lengths (in bases) of the two segments is A and C, where a gap of length B (in bases) is located between the segments, where the gap has a small number of amplicons, such as 2 or fewer amplicons, and has a different copy number than that of the flanking segments;
b) Merge the gap with the two flanking segments by concatenating the gap between the two flanking segments to form a merged segment, where the length of the merged segment is A+B+C.

A span parameter is the number of amplicons that cover the gap region that can be merged with the neighboring long segments. The gap may represent a focal event which can be ignored when calling arm level events. This suppresses the production of small segments. For example, the span parameter may have a value of 2 amplicons. The user may set a value for the span parameter.

In step 114, the cellularities of the gain/loss segments of each arm are tested for a minimum level of cellularity compared to the cellularity of the sample. Cellularity is the fraction (or %) of tumor cells in a sample, also referred to as Tumor Fraction (TF). Heterozygous SNPs are in the normal cells as well as tumor cells, but at different allele frequencies, mediated by the different tumor fractions. Observed copy number levels for a sample that may be a mixture of tumor and normal cells is given by the following:

$$\\ \begin{matrix}CN = TCN * TF + 2 * ( 1 - TF ) & ( 1 )\end{matrix}$$

where CN is observed copy number, TCN is the tumor copy number, and TF is tumor fraction, or cellularity, and 2 represents the diploid copy number. The tumor copy number, TCN, is determined using the ratio of the alternate allele to reference allele for the heterozygous SNPs, as follows:

$$\\ \begin{matrix}Alt / Ref = [ m * TF + 1 * ( 1 - TF ) ] / [ n * TF + 1 * ( 1 - TF ) ] & ( 2 )\end{matrix}$$

$$\\ \begin{matrix}TCN = m + n & ( 3 )\end{matrix}$$

where Alt is the alternate allele copy number, Ref is the reference allele copy number, m is the major copy number and n is the minor copy number. Segments can have different cellularities due to heterogeneity in the sample. The ratio of the cellularity of the gain/loss segment to the cellularity of the whole sample is compared to a threshold, segment_cellularity_threshold. If the ratio of segment cellularity to the whole sample cellularity is less than the segment_cellularity_threshold, then the segment will not be used in further analysis for arm aneuploidy. For a merged segment resulting from step 112, if the ratio of the merged segment's cellularity to the whole sample cellularity is less than the segment_cellularity_threshold, then the merged segment will not be used in further analysis for arm aneuploidy. For example, the segment_cellularity_threshold value may be set to 0.2. The segment_cellularity_threshold represents a minimum level at which the copy numbers can be differentiated between tumor segments and normal segments. Segments having cellularities relative to the sample cellularity that meet the segment cellularity threshold may be further analyzed for arm aneuploidy.

The summing step 120 sums the number of bases in the longest gain/loss segment of the arm to give a total number of bases for the longest segment. In step 122, a minimum length threshold is applied to the longest segment of the as follows:
a) Divide the total number of bases in the longest segment by the number of bases in the arm to give a fraction;
b) Compare the fraction to a minimum fraction threshold, min_fraction;
c) If the fraction is less than the minimum fraction threshold, then the longest gain/loss segment will not be further considered for ARM level change.

For example, the minimum fraction threshold, min_fraction, may be set to a value of 0.9. For min_fraction=0.9, the length of longest gain/loss segment must be at least 90% of the length of the corresponding arm to be further analyzed for an arm level aneuploidy.

The gain filtering step 124 applies a minimum fold change filter to the gain segments. The fold change for a gain segment is compared to a minimum gain threshold for fold difference, min gain_fd. If the fold change for the gain segment is greater than or equal to the minimum gain threshold, then a gain is called for the segment. The fold change multiplied by two is the measured copy number for the segment. For example, a value for the minimum gain threshold is 1.15, which corresponds to a minimum copy number threshold of 2.3 for a gain. Measured copy numbers less than 2.3 compared to the reference copy number, may represent noise in the measurements. The filtering step 124 reduces noise by discounting gain segments that do not have a minimal gain above the reference copy number. So if the calculated copy number is greater than or equal to a minimum threshold above the reference copy number, then the gain segment may be confirmed as having a copy number gain.

The loss filtering step 126 applies a maximum fold change filter to the loss segments. The fold change for a loss segment is compared to a maximum loss threshold for fold difference, max_loss_fd. If the fold change for the loss segment is less than or equal to the maximum loss threshold, then a loss is called for the segment. For example, a value for the maximum loss threshold for fold difference is 0.85, which corresponds to a maximum copy number threshold of 1.7 for a loss segment. Measured copy numbers greater than 1.7 and less than the reference copy number may represent noise in the measurements for copy number of the loss segment. The loss filtering step 126 discounts loss segments that are in the range of 1.7< (copy number)<=ref_cn. If the calculated copy number is less than or equal to a maximum threshold, then loss segment may be confirmed as having a copy number loss.

The gain filtering step 124 and the loss filtering step 126 filter out segments that fall within a range of measured copy numbers given by:

$$\\ \begin{matrix}ref\_cn * max\_loss \_fd < copy number of segment < ref\_cn * min\_gain \_fd & ( 4 )\end{matrix}$$

The gain filtering step 124 and the loss filtering step 126 use thresholds based on the fold changes. Some embodiments may use thresholds based on copy number values. The fold change multiplied by the reference copy number gives the copy number. Some embodiments may use thresholds based on the difference between the copy number and the reference copy number.

Values for the parameters minimum gain threshold for fold difference, min gain_fd, and maximum loss threshold for fold difference, max_loss_fd, may be found empirically by verifying test results with known truth data. The known truth data may result from testing samples using an orthogonal method. For example, an array-based method, such as methods using OncoScan™ arrays (Thermo Fisher Scientific), can be used for testing samples to generate truth data. The parameters minimum gain threshold for fold difference, min gain_fd, and maximum loss threshold for fold difference, max_loss_fd, can be optimized for concordance in results with the truth data set.

In step 128, the p-value is calculated by applying a Student's T-test to the raw copy numbers of the amplicons encompassed by the gain/loss segment. Long gain/loss segments may include multiple amplicons. There may be variations in raw copy numbers among those amplicons, even though they are called as a one long segment and assigned one integer value of the copy number of the segment. The amplicons within the long segment may have different read coverage numbers. If the variance is high for the raw copy numbers of the amplicons of a particular segment, then a gain or loss call for the segment has a low confidence. The p-value of raw copy numbers of the amplicons captures the variance. The p-value is based on the assumption that raw copy numbers have a mean of the reference copy number. The variance of the raw copy numbers of the amplicons within the segment are calculated. The p-value would indicate whether the hypothesis as null, in which case there is no change with respect to a reference copy number, i.e., there is no gain or loss in the particular segment.

In step 130, the p-value is compared to a threshold p-value for a decision to call a gain or a loss for the arm containing the segment. If the p-value is less than or equal to a maximum p-value for a call, max_call pval, then a gain or loss is called for the arm containing the segment. For example, the maximum p-value may have a value of 10-5.

The following rule gives the conditions that must be fulfilled for making an arm gain call, as described with respect to steps 110, 122, 124, and 130:

$$\\ \begin{matrix}cn\_value > ref\_cn AND fraction ( gain\_segment \_length ) >= min\_fraction AND p - value <= max\_call \_pval AND fold\_change >= min\_gain \_fd & ( 5 )\end{matrix}$$

The following rule gives the conditions for making an arm loss call, as described with respect to steps 110, 122, 126, and 130:

$$\\ \begin{matrix}cn\_value < ref\_cn AND fraction ( loss\_segment \_length ) >= min\_fraction AND p - value <= max\_call \_pval AND fold\_change <= max\_loss \_fd & ( 6 )\end{matrix}$$

The copy number for the longest segment in the arm gives the overall copy number for the arm, when the above conditions are fulfilled.

In step 132, the number of arms for which a gain call or a loss call have been made are counted. The total number of arms with gain or loss calls may represent the arm aneuploidy score for the sample. The total number of arms with gain or loss calls may be divided by the number of arms tested in the sample to give a percent version of the arm aneuploidy score. In step 134, the arm aneuploidy score may be reported to the user in a display of results or a file containing results.

The following tables show results of testing samples for arm aneuploidy using the present methods in comparison with an array based method using OncoScan™. The tests included 58 samples analyzed for arm aneuploidy detection by OncoScan™ array based method and 81 test runs on the samples by the methods described herein using the OCA Plus targeted panel. The positive percent agreement (PPA) was calculated for positive arm aneuploidy calls as follows:

$$\\ \begin{matrix}P P A = [ ( predicted true positives ) / ( predicted positives ) ] * 100 & ( 7 )\end{matrix}$$

where predicted true positives resulted from the array based methods using OncoScan™ tests and predicted positives resulted from the present methods using the OCA Plus targeted panel.

The negative percent agreement (NPA) was calculated for negative arm aneuploidy calls as follows:

$$\\ \begin{matrix}N P A = [ ( predicted true negatives ) / ( predicted negatives ) ] * 100 & ( 8 )\end{matrix}$$

where predicted true negatives resulted from the OncoScan™ tests and predicted negatives resulted from the present methods using the OCA Plus targeted panel.

Table 1 shows the PPA and NPA for both positive and negative arm aneuploidy calls. The results show strong concordance in the orthogonal tests for both positive and negative arm aneuploidy calls.

TABLE 1

| | PPA | NPA |
|---|---|---|
| GAIN | 88% | 96.8% |
| LOSS | 81% | 97.6% |

Table 2 shows results of reproducibility tests with the present methods using the OCA Plus targeted panel. Concordance rates for replicate pairs of samples, where a given sample was tested twice using the present methods, were determined for different tumor fractions (TF). The results show increasing concordance rates as the tumor fractions increased.

TABLE 2

| | TF >= | TF >= | TF >= | TF >= | TF >= |
|---|---|---|---|---|---|
| | 40% | 50% | 55% | 60% | 65% |
| Concordance | 0.873 | 0.897 | 0.900 | 0.919 | 0.950 |
| Rate | | | | | |
| Number of | 46 | 34 | 32 | 24 | 21 |
| pairs | | | | | |

The targeted panel and method for determining an arm aneuploidy score described herein provide improvements to the technology over whole genome sequencing (WGS). Sequence assembly methods must be able to assemble and/or map a large number of reads efficiently, such as by minimizing use of computational resources. For example, the sequencing of a human size genome can result in tens or hundreds of millions of reads that need to be assembled before they can be further analyzed. Computer processing of the nucleic acid sequence reads from targeted sequencing reduces computational requirements and memory requirements versus processing for WGS data. For WGS, 3 Gb of the tumor genome would be covered. The data resulting from the nucleic acid sequence reads for WGS would require computations and storage in memory for the nucleic acid sequence reads and variant data. In comparison, the targeted panel that covers approximately 1 Mb of the tumor genome would require substantially fewer computations and substantially less memory for storage of the nucleic acid sequence reads and variant data.

In various embodiments, nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, fluorescent-based detection systems, single molecule methods, etc.

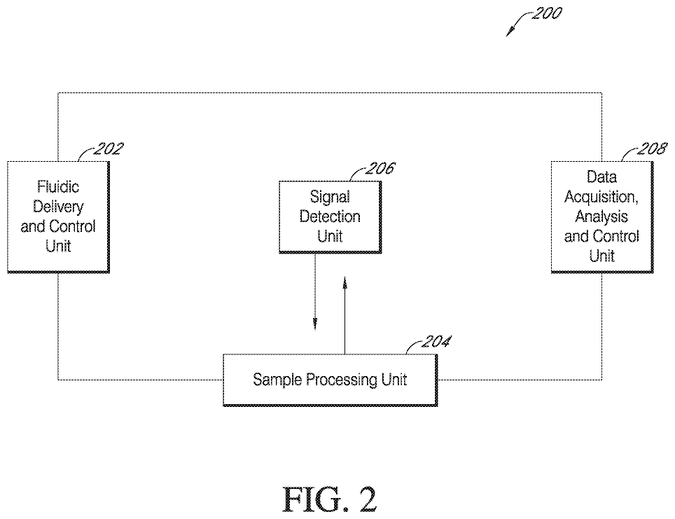

Various embodiments of nucleic acid sequencing platforms, such as a nucleic acid sequencer, can include components as displayed in the block diagram of FIG. 2. According to various embodiments, sequencing instrument 200 can include a fluidic delivery and control unit 202, a sample processing unit 204, a signal detection unit 206, and a data acquisition, analysis and control unit 208. Various embodiments of instrumentation, reagents, libraries and methods used for next generation sequencing are described in U.S. Patent Application Publication No. 2009/0127589 and No. 2009/0026082, each of which incorporated by reference herein in its entirety. Various embodiments of instrument 200 can provide for automated sequencing that can be used to gather sequence information from a plurality of sequences in parallel, such as substantially simultaneously.

In various embodiments, the fluidics delivery and control unit 202 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 204 can include a sample chamber, such as flow cell, a substrate, a micro-array, a multi-well tray, or the like. The sample processing unit 204 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 206 can include an imaging or detection sensor. For example, the imaging or detection sensor can include a CCD, a CMOS, an ion sensor, such as an ion sensitive layer overlying a CMOS, a current detector, or the like. The signal detection unit 206 can include an excitation system to cause a probe, such as a fluorescent dye, to emit a signal. The expectation system can include an illumination source, such as arc lamp, a laser, a light emitting diode (LED), or the like. In particular embodiments, the signal detection unit 206 can include optics for the transmission of light from an illumination source to the sample or from the sample to the imaging or detection sensor. Alternatively, the signal detection unit 206 may not include an illumination source, such as for example, when a signal is produced spontaneously as a result of a sequencing reaction. For example, a signal can be produced by the interaction of a released moiety, such as a released ion interacting with an ion sensitive layer, or a pyrophosphate reacting with an enzyme or other catalyst to produce a chemiluminescent signal. In another example, changes in an electrical current can be detected as a nucleic acid passes through a nanopore without the need for an illumination source.

In various embodiments, data acquisition analysis and control unit 208 can monitor various system parameters. The system parameters can include temperature of various portions of instrument 200, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 200 can be used to practice variety of sequencing methods including ligation-based methods, sequencing by synthesis, single molecule methods, nanopore sequencing, and other sequencing techniques.

In various embodiments, the sequencing instrument 200 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 200 can obtain the sequence information from a single nucleic acid molecule or from a group of substantially identical nucleic acid molecules.

In various embodiments, sequencing instrument 200 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

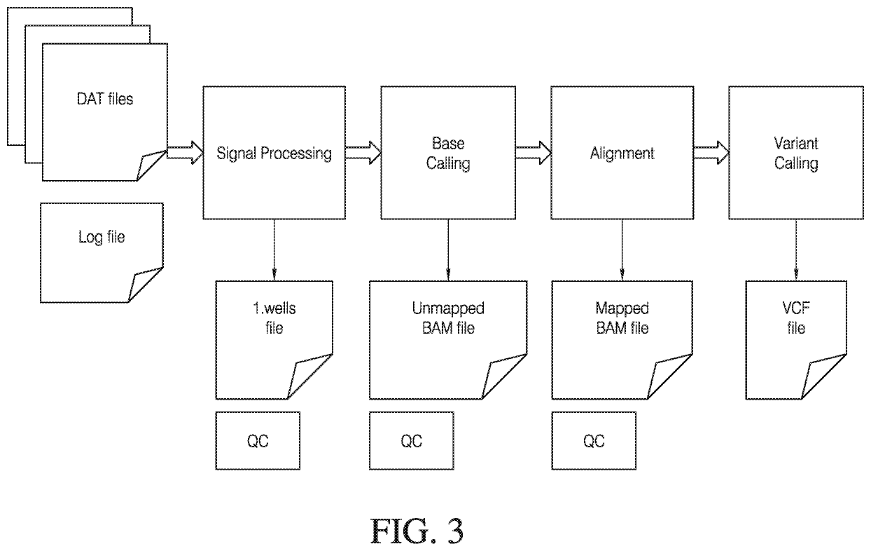

FIG. 3 is a block diagram of an analysis pipeline for signal data obtained from a nucleic acid sequencing instrument. The sequencing instrument generates raw data files (DAT, or .dat, files) during a sequencing run for an assay. Signal processing may be applied to raw data to generate incorporation signal measurement data for files, such as the 1.wells files, which are transferred to the server FTP location along with the log information of the run. The signal processing step may derive background signals corresponding to wells. The background signals may be subtracted from the measured signals for the corresponding wells. The remaining signals may be fit by an incorporation signal model to estimate the incorporation at each nucleotide flow for each well. The output from the above signal processing is a signal measurement per well and per flow, that may be stored in a file, such as a 1.wells file.

In some embodiments, the base calling step may perform phase estimations, normalization, and runs a solver algorithm to identify best partial sequence fit and make base calls. The base sequences for the sequence reads are stored in unmapped BAM files. The base calling step may generate total number of reads, total number of bases, and average read length as quality control (QC) measures to indicate the base call quality. The base calls may be made by analyzing any suitable signal characteristics (e.g., signal amplitude or intensity). The signal processing and base calling for use with the present teachings may include one or more features described in U.S. Pat. Appl. Publ. No. 2013/0090860 published Apr. 11, 2013, U.S. Pat. Appl. Publ. No. 2014/0051584 published Feb. 20, 2014, and U.S. Pat. Appl. Publ. No. 2012/0109598 published May 3, 2012, each incorporated by reference herein in its entirety.

Once the base sequence for the sequence read is determined, the sequence reads may be provided to the alignment step, for example, in an unmapped BAM file. The alignment step maps the sequence reads to a reference genome to determine aligned sequence reads and associated mapping quality parameters. The alignment step may generate a percent of mappable reads as QC measure to indicate alignment quality. The alignment results may be stored in a mapped BAM file. Methods for aligning sequence reads for use with the present teachings may include one or more features described in U.S. Pat. Appl. Publ. No. 2012/0197623, published Aug. 2, 2012, incorporated by reference herein in its entirety.

The BAM file format structure is described in “Sequence Alignment/Map Format Specification,” Sep. 12, 2014 (github.com/samtools/hts-specs). As described herein, a “BAM file” refers to a file compatible with the BAM format. As described herein, an “unmapped” BAM file refers to a BAM file that does not contain aligned sequence read information and mapping quality parameters and a “mapped” BAM file refers to a BAM file that contains aligned sequence read information and mapping quality parameters.

The variant calling step may include detecting single-nucleotide polymorphisms (SNPs), insertions and deletions (InDels), multi-nucleotide polymorphisms (MNPs), and complex block substitution events. In various embodiments, a variant caller can be configured to communicate variants called for a sample genome as a *.vcf, *.gff, or *.hdf data file. The called variant information can be communicated using any file format as long as the called variant information can be parsed and/or extracted for analysis. The variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, each of which is incorporated by reference herein in its entirety.

According to various exemplary embodiments, one or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented using appropriately configured and/or programmed non-transitory machine-readable medium or article that may store an instruction or a set of instructions that, if executed by a machine, may cause the machine to perform a method and/or operations in accordance with the exemplary embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Flash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, R, Python, Pascal, Basic, Fortran, Cobol, Perl, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

### EXAMPLES

Example 1 is a method for determining an arm aneuploidy score for a tumor sample genome, including: selectively amplifying nucleic acid sequences at targeted locations in the tumor sample genome by a targeted panel to generate a plurality of nucleic acid sequence reads; dividing locations in the genome into segments having homogeneous copy numbers using log odds of heterozygous single nucleotide polymorphisms (SNPs) and copy number variation (CNV) log ratios determined for the plurality of nucleic acid sequence reads, wherein the heterozygous SNPs are distributed across the genome; identifying the segments having a gain with respect to a reference copy number as gain segments and the segments having a loss with respect to a reference copy number as loss segments, wherein respective ones of the identified gain/loss segments have locations intersecting respective arms of chromosomes; and for each of a plurality of arms, comparing cellularities of the gain/loss segments to a minimum cellularity relative to a sample cellularity for the tumor sample; summing a number of bases in a longest gain/loss segment to give a total number of bases, wherein the longest gain/loss segment has at least the minimum cellularity; dividing the total number of bases in the longest gain/loss segment by a number of bases in the arm to give a fraction; retaining the longest gain/loss segment if the fraction has a value of at least a minimum fraction threshold; filtering the retained longest gain/loss segment based on gain/loss fold changes; determining a called gain or loss based on copy numbers of amplicons encompassed by the retained longest gain/loss segment; and counting the arms with called gains or losses to give the arm aneuploidy score for the tumor sample.

Example 2 includes the subject matter of Example 1, and further specifies that the step of determining a called gain or loss further comprises calculating a p-value based on the copy numbers of the amplicons encompassed by the retained longest gain/loss segment.

Example 3 includes the subject matter of Example 2, and further specifies that the step of determining a called gain or loss further comprises applying a p-value threshold to the p-value to call a gain or a loss for the arm when the p-value is less than or equal to the p-value threshold, wherein no gain or loss is called for the arm when the p-value is greater than the p-value threshold.

Example 4 includes the subject matter of Example 1, and further specifies that the step of comparing cellularities further comprises, calculating a ratio of the cellularity of the gain/loss segment to the sample cellularity.

Example 5 includes the subject matter of Example 4, and further includes comparing the ratio to a segment cellularity threshold.

Example 6 includes the subject matter of Example 5, and further includes not using the gain/loss segment in further analysis if the ratio is less than the segment cellularity threshold.

Example 7 includes the subject matter of Example 1, and further includes for a given arm, identifying a gap located between two flanking gain/loss segments, wherein the two flanking gain/loss segments have a same copy number and the gap has a different copy number than the copy number of the two flanking gain/loss segments.

Example 8 includes the subject matter of Example 7, and further includes merging the gap with the two flanking gain/loss segments by concatenating the gap with the two flanking gain/loss segments to form a merged segment.

Example 9 includes the subject matter of Example 8, and further specifies that the step of comparing cellularities of the gain/loss segments to a minimum cellularity is applied to the merged segment.

Example 10 includes the subject matter of Example 1, and further specifies that the step of filtering the retained longest gain/loss segment based on gain/loss fold changes further comprises for a given gain segment, applying a minimum fold change filter to the given gain segment.

Example 11 includes the subject matter of Example 10, and further includes comparing a fold change for the given gain segment to a minimum gain threshold.

Example 12 includes the subject matter of Example 11, and further includes confirming the gain for the given gain segment if the fold change for the given gain segment is greater than or equal to the minimum gain threshold.

Example 13 includes the subject matter of Example 1, and further specifies that the step of filtering the retained longest gain/loss segment based on gain/loss fold changes further comprises, for a given loss segment, applying a maximum fold change filter to the given loss segment.

Example 14 includes the subject matter of Example 13, and further includes comparing a fold change for the given loss segment to a maximum loss threshold.

Example 15 includes the subject matter of Example 14, and further includes confirming the loss for the given loss segment if the fold change for the given loss segment is less than or equal to the maximum loss threshold.

Example 16 is a system for determining an arm aneuploidy score for a tumor sample genome, comprising a processor and a memory communicatively connected with the processor, the processor configured to execute instructions, which, when executed by the processor, cause the system to perform a method, including: receiving a plurality of nucleic acid sequence reads generated by selectively amplifying nucleic acid sequences at targeted locations in the tumor sample genome by a targeted panel; dividing locations in the genome into segments having homogeneous copy numbers using log odds of heterozygous single nucleotide polymorphisms (SNPs) and copy number variation (CNV) log ratios determined for the plurality of nucleic acid sequence reads, wherein the heterozygous SNPs are distributed across the genome; identifying the segments having a gain with respect to a reference copy number as gain segments and the segments having a loss with respect to a reference copy number as loss segments, wherein respective ones of the identified gain/loss segments have locations intersecting respective arms of chromosomes; and for each of a plurality of arms, comparing cellularities of the gain/loss segments to a minimum cellularity relative to a sample cellularity for the tumor sample; summing a number of bases in a longest gain/loss segment to give a total number of bases, wherein the longest gain/loss segment has at least the minimum cellularity; dividing the total number of bases in the longest gain/loss segment by a number of bases in the arm to give a fraction; retaining the longest gain/loss segment if the fraction has a value of at least a minimum fraction threshold; filtering the retained longest gain/loss segment based on gain/loss fold changes; determining a called gain or loss based on copy numbers of amplicons encompassed by the retained longest gain/loss segment; and counting the arms with called gains or losses to give the arm aneuploidy score for the tumor sample.

Example 17 includes the subject matter of Example 16, and further specifies that the step of determining a called gain or loss further comprises calculating a p-value based on the copy numbers of the amplicons encompassed by the retained longest gain/loss segment.

Example 18 includes the subject matter of Example 17, and further specifies that the step of determining a called gain or loss further comprises applying a p-value threshold to the p-value to call a gain or a loss for the arm when the p-value is less than or equal to the p-value threshold, wherein no gain or loss is called for the arm when the p-value is greater than the p-value threshold.

Example 19 includes the subject matter of Example 16, and further specifies that the step of comparing cellularities further comprises, calculating a ratio of the cellularity of the gain/loss segment to the sample cellularity.

Example 20 includes the subject matter of Example 19, and further includes comparing the ratio to a segment cellularity threshold.

Example 21 includes the subject matter of Example 20, and further includes not using the gain/loss segment in further analysis if the ratio is less than the segment cellularity threshold.

Example 22 includes the subject matter of Example 16, and further includes for a given arm, identifying a gap located between two flanking gain/loss segments, wherein the two flanking gain/loss segments have a same copy number and the gap has a different copy number than the copy number of the two flanking gain/loss segments.

Example 23 includes the subject matter of Example 22, and further includes merging the gap with the two flanking gain/loss segments by concatenating the gap with the two flanking gain/loss segments to form a merged segment.

Example 24 includes the subject matter of Example 23, and further specifies that the step of comparing cellularities of the gain/loss segments to a minimum cellularity is applied to the merged segment.

Example 25 includes the subject matter of Example 16, and further specifies that the step of filtering the retained longest gain/loss segment based on gain/loss fold changes further comprises for a given gain segment, applying a minimum fold change filter to the given gain segment.

Example 26 includes the subject matter of Example 25, and further includes comparing a fold change for the given gain segment to a minimum gain threshold.

Example 27 includes the subject matter of Example 26, and further includes confirming the gain for the given gain segment if the fold change for the given gain segment is greater than or equal to the minimum gain threshold.

Example 28 includes the subject matter of Examples 16, and further specifies that the step of filtering the retained longest gain/loss segment based on gain/loss fold changes further comprises, for a given loss segment, applying a maximum fold change filter to the given loss segment.

Example 29 includes the subject matter of Example 28, and further includes comparing a fold change for the given loss segment to a maximum loss threshold.

Example 30 includes the subject matter of Example 29, and further includes confirming the loss for the given loss segment if the fold change for the given loss segment is less than or equal to the maximum loss threshold.

Example 31 is a non-transitory computer-readable medium storing instructions that, when executed by a processor, cause the processor to perform a method for determining an arm aneuploidy score for a tumor sample genome, the method comprising: receiving, at the processor, a plurality of sequence reads produced by selectively amplifying nucleic acid sequences at targeted locations in the tumor sample genome by a targeted panel; dividing locations in the genome into segments having homogeneous copy numbers using log odds of heterozygous single nucleotide polymorphisms (SNPs) and copy number variation (CNV) log ratios determined for the plurality of nucleic acid sequence reads, wherein the heterozygous SNPs are distributed across the genome; identifying the segments having a gain with respect to a reference copy number as gain segments and the segments having a loss with respect to a reference copy number as loss segments, wherein respective ones of the identified gain/loss segments have locations intersecting respective arms of chromosomes; and for each of a plurality of arms, comparing cellularities of the gain/loss segments to a minimum cellularity relative to a sample cellularity for the tumor sample; summing a number of bases in a longest gain/loss segment to give a total number of bases, wherein the longest gain/loss segment has at least the minimum cellularity; dividing the total number of bases in the longest gain/loss segment by a number of bases in the arm to give a fraction; retaining the longest gain/loss segment if the fraction has a value of at least a minimum fraction threshold; filtering the retained longest gain/loss segment based on gain/loss fold changes; determining a called gain or loss based on copy numbers of amplicons encompassed by the retained longest gain/loss segment; and counting the arms with called gains or losses to give the arm aneuploidy score for the tumor sample.

Example 32 includes the subject matter of Example 31, and further specifies that the step of determining a called gain or loss further comprises calculating a p-value based on the copy numbers of the amplicons encompassed by the retained longest gain/loss segment.

Example 33 includes the subject matter of Example 32, and further specifies that the step of determining a called gain or loss further comprises applying a p-value threshold to the p-value to call a gain or a loss for the arm when the p-value is less than or equal to the p-value threshold, wherein no gain or loss is called for the arm when the p-value is greater than the p-value threshold.

Example 34 includes the subject matter of Example 31, and further specifies that the step of comparing cellularities further comprises, calculating a ratio of the cellularity of the gain/loss segment to the sample cellularity.

Example 35 includes the subject matter of Example 34, and further includes comparing the ratio to a segment cellularity threshold.

Example 36 includes the subject matter of Example 35, and further includes not using the gain/loss segment in further analysis if the ratio is less than the segment cellularity threshold.

Example 37 includes the subject matter of Example 31, and further includes for a given arm, identifying a gap located between two flanking gain/loss segments, wherein the two flanking gain/loss segments have a same copy number and the gap has a different copy number than the copy number of the two flanking gain/loss segments.

Example 38 includes the subject matter of Example 37, and further includes merging the gap with the two flanking gain/loss segments by concatenating the gap with the two flanking gain/loss segments to form a merged segment.

Example 39 includes the subject matter of Example 38, and further specifies that the step of comparing cellularities of the gain/loss segments to a minimum cellularity is applied to the merged segment.

Example 40 includes the subject matter of Example 31, and further specifies that the step of filtering the retained longest gain/loss segment based on gain/loss fold changes further comprises for a given gain segment, applying a minimum fold change filter to the given gain segment.

Example 41 includes the subject matter of Example 40, and further includes comparing a fold change for the given gain segment to a minimum gain threshold.

Example 42 includes the subject matter of Examples 41, and further includes confirming the gain for the given gain segment if the fold change for the given gain segment is greater than or equal to the minimum gain threshold.

Example 43 includes the subject matter of Example 31, and further specifies that the step of filtering the retained longest gain/loss segment based on gain/loss fold changes further comprises, for a given loss segment, applying a maximum fold change filter to the given loss segment.

Example 44 includes the subject matter of Example 43, and further includes comparing a fold change for the given loss segment to a maximum loss threshold.

Example 45 includes the subject matter of Examples 44, and further includes confirming the loss for the given loss segment if the fold change for the given loss segment is less than or equal to the maximum loss threshold.

What is claimed is:

1. A method for determining an arm aneuploidy score in a tumor sample genome, comprising:
selectively amplifying nucleic acid sequences at targeted locations in the tumor sample genome by a targeted panel to generate a plurality of nucleic acid sequence reads;
dividing locations in the genome into segments having homogeneous copy numbers using log odds of heterozygous single nucleotide polymorphisms (SNPs) and copy number variation (CNV) log ratios determined for the plurality of nucleic acid sequence reads, wherein the heterozygous SNPs are distributed across the genome;
identifying the segments having a gain with respect to a reference copy number as gain segments and the segments having a loss with respect to a reference copy number as loss segments, wherein respective ones of the identified gain/loss segments have locations intersecting respective arms of chromosomes;
for each of a plurality of arms,
comparing cellularities of the gain/loss segments to a minimum cellularity relative to a sample cellularity for the tumor sample;
summing a number of bases in a longest gain/loss segment to give a total number of bases, wherein the longest gain/loss segment has at least the minimum cellularity;
dividing the total number of bases in the longest gain/loss segment by a number of bases in the arm to give a fraction;
retaining the longest gain/loss segment if the fraction has a value of at least a minimum fraction threshold;
filtering the retained longest gain/loss segment based on gain/loss fold changes;
determining a called gain or loss based on copy numbers of amplicons encompassed by the retained longest gain/loss segment; and
counting the arms with called gains or losses to give the arm aneuploidy score for the tumor sample.

2. The method of claim 1, wherein the step of determining a called gain or loss further comprises calculating a p-value based on the copy numbers of the amplicons encompassed by the retained longest gain/loss segment.

3. The method of claim 2, wherein the step of determining a called gain or loss further comprises applying a p-value threshold to the p-value to call a gain or a loss for the arm when the p-value is less than or equal to the p-value threshold, wherein no gain or loss is called for the arm when the p-value is greater than the p-value threshold.

4. The method of claim 1, wherein the step of comparing cellularities further comprises, calculating a ratio of the cellularity of the gain/loss segment to the sample cellularity.

5. The method of claim 4, further comprising comparing the ratio to a segment cellularity threshold.

6. The method of claim 5, further comprising not using the gain/loss segment in further analysis if the ratio is less than the segment cellularity threshold.

7. The method of claim 1, further comprising for a given arm, identifying a gap located between two flanking gain/loss segments, wherein the two flanking gain/loss segments have a same copy number and the gap has a different copy number than the copy number of the two flanking gain/loss segments.

8. The method of claim 7, further comprising merging the gap with the two flanking gain/loss segments by concatenating the gap with the two flanking gain/loss segments to form a merged segment.

9. The method of claim 8, wherein the step of comparing cellularities of the gain/loss segments to a minimum cellularity is applied to the merged segment.

10. The method of claim 1, wherein the step of filtering the retained longest gain/loss segment based on gain/loss fold changes further comprises for a given gain segment, applying a minimum fold change filter to the given gain segment.

11. The method of claim 10, further comprising comparing a fold change for the given gain segment to a minimum gain threshold.

12. The method of claim 11, further comprising confirming the gain for the given gain segment if the fold change for the given gain segment is greater than or equal to the minimum gain threshold.

13. The method of claim 1, wherein the step of filtering the retained longest gain/loss segment based on gain/loss fold changes further comprises, for a given loss segment, applying a maximum fold change filter to the given loss segment.

14. The method of claim 13, further comprising comparing a fold change for the given loss segment to a maximum loss threshold.

15. The method of claim 14, further comprising confirming the loss for the given loss segment if the fold change for the given loss segment is less than or equal to the maximum loss threshold.

16. A system for determining an arm aneuploidy score for a tumor sample genome, comprising a processor and a memory communicatively connected with the processor, the processor configured to execute instructions, which, when executed by the processor, cause the system to perform a method, including:
receiving a plurality of nucleic acid sequence reads generated by selectively amplifying nucleic acid sequences at targeted locations in the tumor sample genome by a targeted panel;
dividing locations in the genome into segments having homogeneous copy numbers using log odds of heterozygous single nucleotide polymorphisms (SNPs) and copy number variation (CNV) log ratios determined for the plurality of nucleic acid sequence reads, wherein the heterozygous SNPs are distributed across the genome;
identifying the segments having a gain with respect to a reference copy number as gain segments and the segments having a loss with respect to a reference copy number as loss segments, wherein respective ones of the identified gain/loss segments have locations intersecting respective arms of chromosomes;
for each of a plurality of arms,
comparing cellularities of the gain/loss segments to a minimum cellularity relative to a sample cellularity for the tumor sample;
summing a number of bases in a longest gain/loss segment to give a total number of bases, wherein the longest gain/loss segment has at least the minimum cellularity;
dividing the total number of bases in the longest gain/loss segment by a number of bases in the arm to give a fraction;
retaining the longest gain/loss segment if the fraction has a value of at least a minimum fraction threshold;
filtering the retained longest gain/loss segment based on gain/loss fold changes;
determining a called gain or loss based on copy numbers of amplicons encompassed by the retained longest gain/loss segment; and
counting the arms with called gains or losses to give the arm aneuploidy score for the tumor sample.

17. The system of claim 16, wherein the step of determining a called gain or loss further comprises calculating a p-value based on the copy numbers of the amplicons encompassed by the retained longest gain/loss segment.

18. The system of claim 17, wherein the step of determining a called gain or loss further comprises applying a p-value threshold to the p-value to call a gain or a loss for the arm when the p-value is less than or equal to the p-value threshold, wherein no gain or loss is called for the arm when the p-value is greater than the p-value threshold.

19. A non-transitory computer-readable medium storing instructions that, when executed by a processor, cause the processor to perform a method for determining an arm aneuploidy score for a tumor sample genome, the method comprising:
receiving, at the processor, a plurality of sequence reads produced by selectively amplifying nucleic acid sequences at targeted locations in the tumor sample genome by a targeted panel;
dividing locations in the genome into segments having homogeneous copy numbers using log odds of heterozygous single nucleotide polymorphisms (SNPs) and copy number variation (CNV) log ratios determined for the plurality of nucleic acid sequence reads, wherein the heterozygous SNPs are distributed across the genome;
identifying the segments having a gain with respect to a reference copy number as gain segments and the segments having a loss with respect to a reference copy number as loss segments, wherein respective ones of the identified gain/loss segments have locations intersecting respective arms of chromosomes;
for each of a plurality of arms,
comparing cellularities of the gain/loss segments to a minimum cellularity relative to a sample cellularity for the tumor sample;
summing a number of bases in a longest gain/loss segment to give a total number of bases, wherein the longest gain/loss segment has at least the minimum cellularity;
dividing the total number of bases in the longest gain/loss segment by a number of bases in the arm to give a fraction;
retaining the longest gain/loss segment if the fraction has a value of at least a minimum fraction threshold;
filtering the retained longest gain/loss segment based on gain/loss fold changes;
determining a called gain or loss based on copy numbers of amplicons encompassed by the retained longest gain/loss segment; and
counting the arms with called gains or losses to give the arm aneuploidy score for the tumor sample.

20. The non-transitory computer-readable medium of claim 19, wherein the step of determining a called gain or loss further comprises calculating a p-value based on the copy numbers of the amplicons encompassed by the retained longest gain/loss segment.
